(19) United States
(12) Patent Application Publication
(10) Pub. No.: US 2013/0337482 A1
(43) Pub. Date: Dec. 19, 2013

(54) ASSAYS AND KITS TO DETERMINE GALACTOCEREBROSIDASE ACTIVITY ON SOLID SUPPORT

(71) Applicant: Gherman Wiederschain, Roslindale, MA (US)

(72) Inventor: Gherman Wiederschain, Roslindale, MA (US)

(73) Assignee: Shire Human Genetic Therapies, Inc., Lexington, MA (US)

(21) Appl. No.: 13/882,414

(22) PCT Filed: Oct. 28, 2011
(86) PCT No.: PCT/US11/58442

§ 371 Date: Aug. 30, 2013

Related U.S. Application Data
(60) Provisional application No. 61/408,429, filed on Oct. 29, 2010.

(51) Int. Cl. C12Q 1/34

(52) U.S. Cl. CPC C12Q 1/34
USPC 435/18

(57) ABSTRACT

The present invention discloses compositions, methods, assays and kits which provide for rapid, high-throughput and sensitive assays useful for detecting the activity of galactocerebrosidase (GALC) in a test sample. The methods, assays and kits of the present invention provide useful diagnostic tools which may be used to identify subjects suspected of having an enzyme deficiency and to evaluate the efficacy of enzyme replacement therapy.

### RELATED APPLICATIONS

This application claims the benefit of U.S. Provisional Application No. 61/408,429, filed Oct. 29, 2010. The entire teachings of the above application are incorporated herein by reference.

### BACKGROUND OF THE INVENTION

Globoid cell leukodystrophy (GLD) or Krabbe disease is a rare and often fatal neurological lysosomal storage disorder caused by mutations in the gene encoding galactocerebrosidase (or galactosylceramidase) (GALC). GALC is an enzyme which is responsible for removing galactose from ceramide derivatives. Deficiency in GALC activity causes an abnormal accumulation of cytotoxic psychosine, particularly in myelin-forming cells, which leads to demyelination in both the central nervous system and peripheral nerves during early developmental stages.

Advances in molecular biology have supported the development of recombinant biological agents (e.g., recombinant proteins and/or enzymes) which are useful for modulating protein or enzyme activity or nucleic acid expression. For example, such recombinantly prepared proteins and/or enzymes may be therapeutically administered to humans who demonstrate an endogenous deficiency of such protein and/or enzyme. The diagnosis of such endogenous deficiencies, as well as the in vitro production of recombinant biological agents both require an analytically sensitive assay which is capable of rapidly assessing and quantifying enzymatic (e.g., GALC) activity.

Generally, assays used to assess enzymatic activity involve contacting a biological sample or a recombinantly prepared enzyme with a substrate with which the enzyme of interest is known to predictably react. Enzymatic activity may be subsequently measured by either evaluating the depletion of the substrate and/or the yield of an enzymatic product over time. Different methods of measuring the concentrations of substrates and/or products exist and many enzymes can be assayed in several different ways, many of which are time-consuming. For example, GALC enzymatic activity has been measured through a variety of biochemical assays which are based largely on radiolabeled galactocerebroside or histological assays. (See, e.g., Raghavan, S., et al. Biochim Biophys Acta, 877: 1-8 (1986) and Dolcetta, D., et al., J Neurosci Res, 77: 462-4 (2004)). The utility of such assays is often limited, for example, due to poor limits of detection, the relatively large amounts of protein or enzyme required to perform such assays, and the length of time required to perform such assays. Furthermore, assays available to analyze enzymatic activity are frequently performed in a test tube which may prove especially cumbersome and time consuming, for example, due to the handling of hazardous waste and/or the immobilization of substrates onto solid supports. Such analyses may be further complicated by the performance of multiple steps or reactions, each often performed on a different solid support.

There remains a need for improved methods and high-throughput assays for the routine analysis of enzymatic activity, and in particular GALC activity. There is also a need for improved methods and sensitive, high-throughput assays which are capable of being performed quickly, accurately and preferably in a single solid support.

### SUMMARY OF THE INVENTION

The present invention relates to methods, assays and kits which are useful for the high-throughput determination of enzymatic activity of a test sample. The present invention also provides methods and assays which facilitate the rapid identification and quantification of enzymatic activity in a test sample. Generally, such methods, assays and kits comprise the steps of contacting a test sample with a substrate and using routine means (e.g., absorption or fluorescence spectroscopy techniques) to quantify the product of an enzymatic reaction, or alternatively the depletion of a substrate. In a preferred embodiment of the present invention, the methods and assays may be performed using a low volume or amount of a test sample or biological sample (e.g., 100 μg, 75 μg, 50 μg, 40 μg, 30 μg, 25 μg, 20 μg, 15 μg, 10 μg, 5 μg, 1 μg or less of tissue homogenate or pure enzyme or protein). The methods, assays and kits of the present invention contemplate the use of substrates which are known to predictably react with an enzyme whose presence is suspected in a test sample. In some embodiments the methods, assays and kits of the present invention provide useful tools to measure the presence of GALC in a test sample, or alternatively the principles presented herein can be applied generally to determine the presence of any particular enzyme in a test sample. The methods, assays and kits of the present invention also provide useful tools to facilitate the identification or diagnosis of subjects suspected of having an enzyme deficiency (e.g., globoid cell leukodystrophy), or alternatively facilitate monitoring of the efficacy of treatments (e.g., enzyme replacement therapy) administered to such subjects.

The methods, assays and kits of the present invention provide tools which are useful to visually, colorimetrically, fluorometrically and/or chemically distinguish the presence or absence of a predicted enzymatic reaction, and are preferably capable of quantitatively determining the presence of an enzyme (e.g., galactocerebrosidase) in a test sample using routine means (e.g., absorption or fluorescence spectroscopic techniques). For example, in one embodiment of the present invention, enzymatic activity, such as the hydrolysis of a substrate by an enzyme may yield a fluorescently-detectable signal which can be measured using fluorescence spectroscopy and thus provide means of quantitatively assessing the presence of such enzyme. The methods, assays and kits may optionally be useful for the rapid, high-throughput detection of an enzyme as a diagnostic marker, predictor or identifier of disease (e.g., diagnosis of galactocerebrosidase deficiencies by evaluating a test sample obtained from a human). In particular, the methods, assays and kits of the present invention are capable of providing rapid and high-throughput identification of GALC deficiency or the diagnosis and monitoring of globoid cell leukodystrophy (GLD) or Krabbe disease.

Another embodiment of the present invention relates to methods and assays which are useful for measuring the activity of enzymes, for example, GALC in a solid support (e.g., a 96-well microplate). Such methods and assays comprise the steps of determining GALC activity by reacting a test sample with a fluorogenic substrate (e.g., 6-hexadecanoylamino-4-methylumbelliferyl-β-D-galactopyranoside or HMGal). The reaction is subsequently halted by contacting the reactants with a stop-buffer or an anti-catalyst (e.g., taurocholic acid, glycine and/or sodium dodecylsulphate) to enable quantification of the applicable reactants. The present invention contemplates determining and/or quantifying GALC in the test sample by, for example, quantification of a fluorogenic detectable signal (e.g., 6-decanoylamino-4-methylumbelliferone) which is liberated upon hydrolysis of HMGal by GALC during the preceding reaction. In a preferred embodiment, the assay is performed in a single solid support (e.g., a 96-well microplate).

In a preferred embodiment, GALC activity is determined as a function of 6-decanoylamino-4-methylumbelliferone production in the solid support, for example using known spectroscopic techniques (e.g., using spectroflurometer at an excitation wavelength of about 385 nm and an emission wavelength at 450 nm) to determine the presence of 6-decanoylamino-4-methylumbelliferone. In a preferred embodiment, the assays and methods of the present invention are performed in a single solid support (e.g., a 96-well microplate).

Also contemplated are kits for determining GALC activity in a test sample. Such kits may comprise at least one solid support, at least one substrate and an anti-catalyst. The kits of the present invention may further comprise a buffer (e.g., citric acid, sodium phosphate and combinations thereof).

The above discussed and many other features and attendant advantages of the present invention will become better understood by reference to the following detailed description of the invention when taken in conjunction with the accompanying examples. The various embodiments described herein are complimentary and can be combined or used together in a manner understood by the skilled person in view of the teachings contained herein.

### BRIEF DESCRIPTION OF THE DRAWINGS

FIG. 1 represents the calibration curve for 6-Hexadecanoylamino-4 methylumbelliferyl (HM) standards using the microplate assay described in Example 1.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and are not intended to be limiting.

Provided herein are novel methods, assays and kits which are useful for the quick and accurate analysis of the enzymatic activity of a test sample. The present invention relies upon the intrinsic properties of an enzyme (e.g., GALC) whose presence is suspected in a test sample as the means of detecting and/or quantifying such enzyme. The invention contemplates contacting the enzyme whose presence is suspected in the test sample with substrates and/or additional reactants, such that if the enzyme is present in the test sample, such enzyme will catalyze a predicted reaction, the result of which is the production or destruction of a detectable signal. In a preferred embodiment, the methods and assays of the present invention are based on the quantification of a detectable signal (e.g., specified chromogenic or fluorogenic reagents) where the all steps (e.g., incubation, color developing, heating, absorbance reading and the determination of enzymatic activity) are performed in a solid support (e.g., a 96-well microplate).

The assays and methods of the present invention provide additional advantages relative to traditional assays, such as for example, assays which rely on the use of radiolabeled galactocerebroside as a substrate to detect or quantify enzymatic activity. For example, the assays and methods of the present invention may be performed in few steps and can be completed within a relatively short period of time (e.g., in twenty-four hours, eighteen hours, twelve hours, eight hours, six hours, four hours, two hours, sixty minutes, thirty minutes, or less). On the other hand, traditional assays used for detection and/or quantification of an enzymatic reaction proceed over the course of days (e.g., assays relying on radiolabeled substrate may require dozens of steps and require two or more working days to complete).

The present invention contemplates the detection and/or quantification of a particular enzyme in a test sample based upon such enzyme's ability to catalyze a particular reaction. In particular, the present invention provides a specific and sensitive assay which may be use to detect enzymatic activity in a test sample, or alternatively for the biochemical diagnosis of globoid cell leukodystrophy (GLD). As used herein, the phrase “enzymatic activity” refers to an enzyme's ability to catalyze a repeatable biochemical reaction, for example, when contacted with a substrate with which such enzyme is known to react. In a preferred embodiment of the present invention, the enzymatic activity of an enzyme may be exploited to confirm the presence or absence of such enzyme in a particular test sample. For example, many enzymes have known and repeatable catalytic activity which may be enhanced under certain conditions (e.g., in the presence of a substrate) and the present inventions exploit such catalytic activity as a means of detecting the enzyme. As used herein, the term “catalyzes” means to accelerate the rate of a reaction. Such a reaction may be catalyzed by a substance which remains chemically unchanged by that reaction.

The methods, assays and kits of the present invention are particularly useful for determining the enzymatic activity of the enzyme galactocerebrosidase (GALC). GALC (EC 3.2.1.46) is a lysosomal enzyme responsible for catalyzing the hydrolysis of galactosylceramide, a major lipid in myelin, kidney, and epithelial cells of the small intestine and colon. (Chen, Y Q., et al., Hum. Molec. Genet. 2: 1841-1845, (1993)). A deficiency of GALC causes the neurodegenerative lysosomal storage disease GLD. (See, e.g., Wenger, D., et al. In: The Metabolic and Molecular Bases of Inherited Disease (8th edition), Scriver C., Beaudet A., Sly W., Valle D. (Eds.), McGraw-Hill, New York, 3669-3694 (2001)). Although the methods, assays and kits of the present invention are useful for determining the activity of the enzyme galactocerebrosidase for the biochemical diagnosis of GLD, the inventions and concepts described herein are generally applicable to the detection of other enzymes.

Enzymatic activity may be measured by routine means known to one of ordinary skill in the art (e.g., colorimetric, spectrophotometric, fluorometric or chromatographic detection assays) by determining, for example the consumption or depletion of substrate and/or the production of a product over time. In accordance with the present invention, substrate depletion of about 5%, 10%, 20%, 30%, 40%, 50% or more, or preferably about 55%, 60%, 65%, 70%, 75%, 80%, 85%, 90%, 95%, 99% or more relative to the amount of substrate introduced may be indicative of enzymatic activity. Alternatively, following contacting an enzyme with a substrate, a relative increase in the formation of a product, or the conversion of that substrate to a product, in each case of about 5%, 10%, 20%, 30%, 40%, 50% or more, or preferably about 55%, 60%, 65%, 70%, 75%, 80%, 85%, 90%, 95%, 99%, or more preferably 100% or more, may be indicative of enzymatic activity.

The methods, assays and kits of the present invention contemplate the selection of appropriate enzyme substrates and/or reactants to determine enzymatic activity. As such, an understanding of enzyme kinetics and in particular the catalytic properties of the enzyme(s) being evaluated are required to practice the methods described herein. Knowledge of the substrate specificity of the enzyme whose presence is suspected in a test sample can enable the identification of such enzyme. The properties of an enzyme, such as for example, Michaelis-Menton constants (Kₘ) and/or turnover numbers (K_{cat}) as they relate to a particular substrate, provide the basis for evaluating the sensitivity of an enzyme for one or more substrates and provide information regarding the reproducibility of the methods and assays contemplated by the present inventions.

As used herein, the terms “react” and “reaction” are used in their broadest senses and refer to enzymatic, chemical, physical and/or biological processes (e.g., hydrolysis) which alter or transform one or more of the participating reactants. For example, test samples with known enzymatic activity (e.g., GALC) may be expected to react with a particular substrate (e.g., 6-hexadecanoylamino-4-methylumbelliferyl-β-D-galactopyranoside) which, under certain conditions will transform one or more of the reactants (e.g., GALC catalyzes the transformation of 6-hexadecanoylamino-4-methylumbelliferyl-β-D-galactopyranoside to 6-decanoylamino-4-methylumbelliferone). In an enzymatic reaction, a reactant may be expended in the reaction to yield a product. In accordance with the present invention, a determination of the depletion of one or more reactants and/or the yield of one or more products will signal an enzymatic reaction, and accordingly the presence of the enzyme in the test sample.

The terms “contact” or “contacting” mean bringing two or more moieties together, or within close proximity of one another such that the moieties may react. For example, in one embodiment of the present invention, contacting an enzyme with its corresponding substrate may be expected to cause an expected reaction.

To evaluate and quantify enzymatic activity, the enzymatic reaction should preferably be halted. The present invention contemplates the addition of an anti-catalyst or stop-buffer to the enzymatic reaction to slow, stop or terminate such enzymatic, chemical, or biological reaction. For example, the addition of an anti-catalyst to an ongoing enzymatic reaction would be expected to slow or stop the catalytic activity of the enzyme. Suitable anti-catalysts contemplated by the invention, include, for example, glacial acetic acid, phosphoric acid, sulfuric acid and combinations thereof.

As used herein, the term “substrate” refers to a molecule, complex, material, substance or reactant upon which an enzyme acts (e.g., chemically or biologically). Generally, the substrate participates in a biochemical enzymatic reaction due to the enzymatic activity of the enzyme. Preferably, the substrates contemplated by the present are specific for an enzyme present in a test sample. For example, 6-hexadecanoylamino-4-methylumbelliferyl-β-D-galactopyranoside is a substrate which is specific for GALC. When contacted with 6-hexadecanoylamino-4-methylumbelliferyl-β-D-galactopyranoside, the enzyme GALC would be expected to catalyze a reaction whereby 6-hexadecanoylamino-4-methylumbelliferyl-β-D-galactopyranoside acid is transformed to the product 6-decanoylamino-4-methylumbelliferone. Substrates useful in the practice of the methods of the present inventions can be native or modified. Modified substrates useful in the invention retain the ability to be acted upon by a corresponding enzyme. Exemplary modifications suitable for substrates include, for example, labeling to confirm the presence or absence of enzymatic activity (e.g., fluorogenic substrates).

As used herein, the phrase “test sample” is used in its broadest sense and means any solid or liquid preparation suspected of having enzymatic activity. Test samples are preferably obtained from biological media or materials, including biologically or recombinantly derived media which may contain, among other things, naturally occurring or recombinantly prepared peptides, polypeptides or proteins, enzymes, lipid or carbohydrate molecules, or glycosylated proteins or enzymes, or other samples obtained from a recombinant media, including any fractions thereof. The test samples contemplated by the present invention may be obtained from in-process or “dirty” biological systems, for example, those obtained during the preparation of a recombinant enzyme. In a preferred embodiment, test samples include biological samples (e.g., samples obtained for diagnostic purposes) such as, for example samples comprising tissue, whole blood, serum, plasma, cell lysates, lymphatic fluid, saliva, cerebrospinal fluid, synovial fluid, urine, nasal secretion, and other bodily fluids. The methods, assays and kits of the present invention advantageously require a limited amount of protein or enzymes in the test sample. In a preferred embodiment of the present invention, the methods and assays may be performed using a low volume or amount of a test sample or biological sample (e.g., 100 μg, 75 μg, 50 μg, 40 μg, 30 μg, 25 μg, 20 μg, 15 μg, 10 μg, 5 μg, 1 μg or less of tissue homogenate or pure enzyme or protein). In another embodiment the amount of enzyme (e.g., GALC) required to perform the methods and assays of the present invention does not exceed 10-20 μg of tissue homogenate, or 1-5 μg of the pure enzyme. In preferred embodiments, the test sample is obtained from a biological source, such as cells in culture or a tissue sample from an animal or microorganism, most preferably, a human. A suitable test sample may be obtained from lysates of selected microorganisms and prepared in accordance with the present invention. To determine the enzymatic activity of a test sample, such test sample is contacted with selected substrates and/or reactants such that if the enzyme of interest is present in the test sample the presence of a detectable signal will indicate enzymatic activity.

Detection and/or quantification of enzymatic activity are preferably determined by measuring the presence of one or more chemically, fluorometrically or colorimetrically detectable signals. As used herein, the phrase “detectable signal” is used in its broadest sense to refer to any indicator of enzymatic activity. Preferably, detectable signals are measurable using routine means. The presence of the detectable signal, and where appropriate its measurement, facilitate the determination and/or quantification of enzymatic activity when used in accordance with the present invention. Preferably, the presence or absence of a detectable signal correlates to the presence or absence, respectively, of enzymatic activity in the test sample.

In one embodiment of the present invention, a detectable signal may be produced by the addition of one or more chromogenic reagents to an enzymatic reaction under appropriate conditions such that the chromogenic reagent will conjugate to one or more of the participating reactants, or alternatively to the product of a reaction. The conjugation of the chromogenic reagent to the reactant or the product thus provides a detectable signal which will enable detection and/or quantification of the reactant or product to which it conjugates. In a particular embodiment of the present invention, the addition of a chromogenic reagent to the enzymatic reaction may conjugate with a reactant to form a detectable signal, that will in-turn enable quantification of such reactant in the reaction. In one embodiment, the reduction in the amount, or the absence of such a detectable signal may be indicative of enzymatic activity. Alternatively, in another embodiment the chromogenic reagent may be capable of facilitating the detection of an enzymatic product (e.g., by conjugating to the product of an enzymatic reaction).

In another embodiment of the present invention, the use of a fluorogenic substrate (e.g., HMGal) may liberate a detectable signal (e.g., 6-decanoylamino-4-methylumbelliferone) when contacted with an enzyme (e.g., GALC) with which it is known to react. The exposure of such a fluorogenic substrate to an enzyme, for example an enzyme known to predictably hydrolyze it under appropriate conditions, will liberate a detectable signal which is indicative of enzymatic activity. As used herein, the term “fluorogenic” refers to a state or condition of having the capability to be fluorescent. As used herein, the term “fluorogenic substrate” refers to a non-fluorescent or weakly-fluorescent enzyme substrate that becomes more fluorescent (e.g., at least about 2, 4, 6, 10, 20, 50 or 100 times more fluorescent) upon the occurrence of an enzymatic, chemical, biochemical, physical and/or other similar transformative event. For example, in one embodiment of the present invention, upon enzymatic hydrolysis of the fluorogenic substrate HMGal the detectable signal 6-decanoylamino-4-methylumbelliferone is liberated. (See, e.g., Wiederschain, G., et al., Carbohydrate Res. 224; 255-272 (1992); and Wiederschain, G., et al., Clin. Chim Acta 205; 87-96 (1992), which are incorporated by reference herein in their entirety). The liberated 6-decanoylamino-4-methylumbelliferone may then be detectable using routine means (e.g., a spectrofluorometer) by an excitation wavelength of about 360-385 nm, whereupon the product emits at about 450-460 nm. In accordance with the present invention, detection of 6-decanoylamino-4-methylumbelliferone is indicative of hydrolysis of the fluorogenic substrate HMGal and corresponds to the presence of enzymatic activity in the test sample. Conversely, the absence of 6-decanoylamino-4-methylumbelliferone is indicative of the lack of hydrolysis of the fluorogenic substrate and corresponds to the absence of enzymatic activity in the test sample.

The high-throughput methods, assays and kits of the present invention contemplate the determination of enzymatic activity, including the steps of incubation and final absorbance reading, in a single solid support (e.g., a 96-well microplate), thus avoiding issues relating to, for example, multiple transfers of reactants or the reaction media. The methods, assays and kits of the invention permit real-time analysis of enzymatic activity while providing enhanced convenience and maintaining sensitivity. By determining enzymatic activity in a single solid support, the present invention provides quick and accurate means of assessing enzymatic activity. The high-throughput assays, methods and kits of the present invention are particularly distinguishable from traditional test tube-based assays, which often require the performance of multiple steps and multiple transfers between solid supports. As the phrase is used herein, “solid support” refers to an inert solid or semi-solid material in which, or on which the enzymatic activity of a test sample may be assessed in accordance with the assays and methods of the present invention. Typical solid supports include, for example, beads, tubes, chips, resins, plates, microplates, wells, films, and sticks. The solid supports may comprise various materials, for example, plastic, glass, ceramic, silicone, metal, cellulose, gels, polystyrene, polyester, and dextran. In a preferred embodiment, the solid support contemplated by the present invention is a standard multiple-well microplate (e.g., a standard polystyrene 96-well microplate, flat bottom with low evaporation lid and well volume.)

The present invention also relates to kits which are useful for determining enzymatic activity of a test sample. Such kits preferably comprise reagents necessary to initiate an enzymatic reaction and facilitate the determination of enzymatic activity. For example, one embodiment of the present invention contemplates kits for determining the presence of GALC in a test sample, wherein such kits may comprise a solid support, 6-hexadecanoylamino-4-methylumbelliferyl-β-D-galactopyranoside and an anti-catalyst. In one embodiment, the components of such kits are integrated into a single solid support such that the determination of enzyme activity is performed in that solid support. Preferably, the kits of the present invention further comprise suitable colorimetric standards which are useful for measuring enzymatic activity in solid support.

The identification and quantification of the detectable signals described herein (e.g., the formation of colored products or the detection of color or the absorption of light) may be performed by any suitable known means. The simplest is visual observation of color development or color change. Alternatively, embodiments of the present invention requiring quantitative measurement will best be performed by spectrofluorometry. Choice of the detection device will be governed by the intended application and considerations of cost, convenience, and whether creation of a permanent record is required.

Detection of molecules by fluorescence has several advantages compared to alternative detection methods. Fluorescence provides an unmatched sensitivity of detection, as demonstrated by the detection of single molecules using fluorescence. (Weiss, S. Science 283: 1676-1683 (1999)). Detection of fluorescence, changes in fluorescence intensity or changes in emission spectra can be easily achieved by the selection of specific wavelengths of excitation and emission. Fluorescence provides a real-time signal, allowing real-time monitoring of processes and real-time cellular imaging by microscopy. (Lakowicz, J. R. Principles of Fluorescence Spectroscopy, Kluwer Academic Plenum Press, New York, 1999, which is herein incorporated by reference). Additionally, well-established methods and instrumentation for high-throughput detection of fluorescence signals exist in the art. (Hill J., et al., Methods in Enzymol. 278: 390-416 (1997)).

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

### EXAMPLE 1

### Determining Galactocerebrosidase (GALC) Activity in 96-Well Plate Format

The present experiments were designed to provide a specific, sensitive high-throughput assay for determining galactocerebrosidase (GALC) activity of a test sample in a 96-well microplate. Generally, GALC activity was assessed by reacting GALC with a fluorogenic substrate capable of liberating a detectable signal upon hydrolysis by GALC.

### Materials and Equipment

Spectrofluorometer (SpectraMaxM2, Molecular Devices);
Micro-plate Incubator/Shaker: (MaxQTM Mini 4000, Barnstead International);
N-Evap nitrogen evaporator with 24 ports and thermostatically controlled water bath (Organomation Associates Inc.);
Black Microtiter Fluorometric 96-well flat bottom Microplate 450 μl/well (Thermo Electron Corporation, Cat.: 9502867);
Sonicator for cells and tissues: Branson Sonifier 250 with tip for 1 ml Eppendorf tube;
Sonicator for substrate mixture preparation (Aquasonic, Model 50 HT, VWR Scientific);
Pipettes for various volumes;
USA Scientific cap strips (Cat. #1494-1400);
Pyrex glass test tubes with Teflon-faced screw caps; and
Aluminum foil

### Assay Protocol

### Sample Preparation

Fluorogenic substrate 6-hexadecanoylamino-4-methylumbelliferyl-β-D-galactopyranoside (HMGal) (Moscerdam) substrate-solution was prepared by dissolving 1 mg HMGal in 15 ml of CHCl₃/MeOH (2:1). Taurocholic acid sodium salt hydrate (TCNa) (Sigma T4009-10) 10 mg/ml solution was prepared using CHCl₃/MeOH mixture (2:1). A total substrate mixture (TSM) was then prepared based on the following calculation per well: 50 μl of HMGal solution, 50 μl of oleic acid solution and 25 μl of solution of TCNa.

A standard solution containing 6-hexadecanoyl-4-methylumbelliferone sodium (6-HM) (Glycosynth Cat. #62073) was prepared by dissolving 1 mg 6-HM in 100 ml of isopropanol.

Test Sample Enzyme extracts (e.g., leukocytes, fibroblast cell pellets or autopsy material of various tissues from animal or human) needed to be homogenized in Potter-Elvehjem homogenizer with Teflon pestle using 0.1-0.2 M citric-phosphate buffer, pH 4.5 (tissue/buffer ratio of approximately 1:10) in ice water-bath. For full GALC solubilization homogenates need to be sonicated. During sonification the Eppendorf tube with homogenate was kept in a beaker with ice water to prevent overheating and enzyme inactivation. The supernatants obtained after homogenates centrifugation at 9300 RCF (10000 rpm) for 15 min at 0-4° C. were used as the enzyme source.

A reaction substrate buffer (RSB), was prepared using Mcllvains phosphate/citric-acid buffer. After mixing the RSB was adjusted under pH-meter control to pH 4.5 with citric acid or sodium phosphate as needed.

### Assay Procedure

125 μl of TSM solution was added to each of the wells on the plate in triplicate for each unknown test sample plus triplicate for TSM-blank (instead of volume of enzyme solution used same volume of RSB);
10 μl of preliminary diluted test sample extracts from cells/tissues were added to the bottom of the wells in duplicate/triplicate following fast addition of 90 μl of TSM;
Blank wells set up in duplicate/triplicate using 90 μl of TSM and 10 μl of reagent buffer instead of enzyme source;
Approximately 20 wells were reserved for 6-HM standards and 2 wells for blank containing 100 μl of buffer;
The microplate was covered with cap strips and placed on plate shaker for 1 hr at 37° C. with continued shaking with setting 100 rpm;
During plate incubation various concentrations of HM standard solutions were prepared starting from dilution of 6-HM stock solution (10 μg/ml) in 2 fold by reaction buffer and 5 serial dilutions (by a factor of 2) from the highest concentration (1,165.5 pmol/100 μL) to the lowest (18.2 pmol/100 μL) in duplicates for a standard curve according to the chart below:

| | | | | | Volume of Citric- |
|---|---|---|---|---|---|
| Well's location | HM-Concentration | | | Volume and Source of | Phosphate Buffer (μL) |
| on the plate | ng/μl | ng/well | pmol/well | HM-Solution (μL) | for dilution |
| A1/A2 | 5.0 | 500 | 1,165.5 | 300 from Stock sol. | 300 |
| B1/B2 | 2.5 | 250 | 582.8 | 300 from A | 300 |
| C1/C2 | 1.25 | 125 | 291.4 | 300 from B | 300 |
| D1/D2 | 0.625 | 62.5 | 145.7 | 300 from C | 300 |
| E1/E2 | 0.313 | 31.5 | 73.0 | 300 from D | 300 |
| F1/F2 | 0.156 | 15.6 | 36.4 | 300 from E | 300 |
| G1/G2 | 0.078 | 7.8 | 18.2 | 300 from E | 300 |
| H1/H2 | 0 | 0 | 0.0 | 0 | 0 |

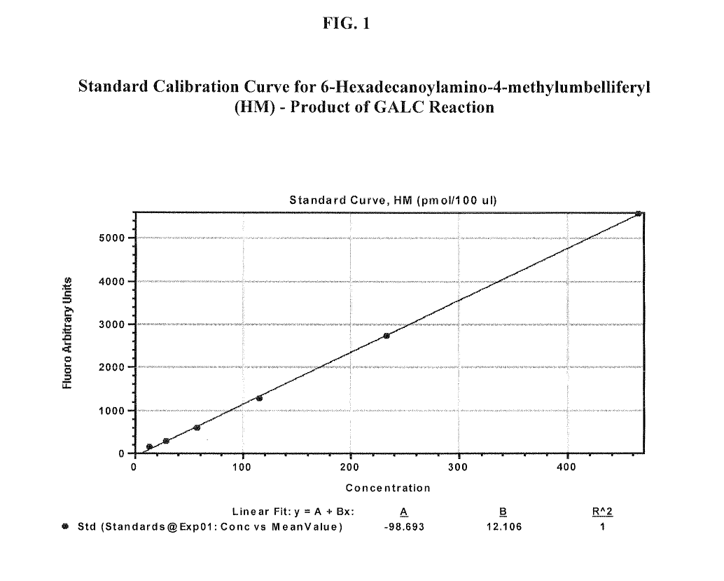

After incubation of test samples 100 μl from each of the standards concentrations was added to the empty wells;
300 μl of anti-catalyst solution (prepared by dissolving 15.01 g glycine in approximately 900 mL DI water and adding 20 ml of 10% SDS and 2 ml of Triton X-100) was added to each well including wells with all blanks and standards; and
The plate was the read using spectrofluorometer with excitation wavelength at 385 nm and emission wavelength at 450 nm with preliminary plate shaking for 5 seconds. GALC activity was calculated per pmol of hydrolyzed HMGal per mg protein per 1 hr using value of 6-HM quantities in pmol from an HM-standard curve (FIG. 1) as enzyme reaction product equivalent of hydrolyzed HMGal.

### Results

The data obtained in this microplate assay demonstrated good sensitivity and linearity of the HM standards (in the pmol range) for detecting and analyzing GALC activity in normal tissue. The assay and the methods described were successfully implemented during GALC purification procedures, cell culture lysates production and for other GALC sources, supporting the conclusion that the methods and assays are also useful for routine high-throughput diagnostic or screening procedures, as well as providing sensitive assays useful to monitor or evaluate the efficacy of enzyme replacement or gene therapies administered to subjects for the treatment of GLD.

What is claimed is:

1. A method of measuring galactocerebrosidase activity of a test sample in solid support, comprising: (i) contacting said test sample with a fluorogenic substrate; (ii) contacting the reactants of step (i) with an anti-catalyst; and (iii) measuring the production of a detectable signal in said solid support.

2. The method of claim 1, wherein said fluorogenic substrate comprises 6-hexadecanoylamino-4-methylumbelliferyl-β-D-galactopyranoside.

3. The method of claim 1, wherein said fluorogenic substrate is hydrolyzed by galactocerebrosidase.

4. The method of claim 3, wherein upon hydrolysis of said fluorogenic substrate a detectable signal is produced.

5. The method of claim 4, wherein said detectable signal comprises 6-decanoylamino-4-methylumbelliferone.

6. The method of claim 1, wherein said galactocerebrosidase activity is measured by detection of a detectable signal using a spectrofluorometer.

7. The method of claim 6, wherein said detectable signal has an excitation wavelength of about 385 nm.

8. The method of claim 6, wherein said detectable signal has an emission wavelength of about 450 nm.

9. The method of claim 4, wherein said galactocerebrosidase activity of a test sample is directly proportional to the production of a detectable signal.

10. The method of claim 1, wherein said anti-catalyst is selected from the group consisting of taurocholic acid, glycine, sodium dodecylsulphate and combinations thereof.

11. The method of claim 1, wherein said solid support is selected from the group consisting of beads, tubes, chips, resins, plates, wells, films, and microplates.

12. The method of claim 1, wherein said solid support is selected from the group consisting of plastic, glass, ceramic, silicone, metal, cellulose, gels, polystyrene, polyester, and dextran.

13. The method of claim 1, wherein said solid support is a 96-well microplate.

14. The method of claim 1, wherein said steps (i), (ii) and (iii) are performed in one solid support.

15. A kit for measuring galactocerebrosidase activity in a test sample, comprising: (i) at least one solid support; (ii) a fluorogenic substrate; and (iii) an anti-catalyst.

16. The kit of claim 15, wherein said fluorogenic substrate comprises 6-hexadecanoylamino-4-methylumbelliferyl-β-D-galactopyranoside.

17. The kit of claim 15, wherein upon hydrolysis of said fluorogenic substrate a detectable signal is produced.

18. The kit of claim 17, wherein said detectable signal comprises 6-decanoylamino-4-methylumbelliferone.

19. The kit of claim 15, wherein said galactocerebrosidase activity is measured by detection of a detectable signal using a spectrofluorometer.

20. The kit of claim 19, wherein said detectable signal has an excitation wavelength of about 385 nm.

21. The kit of claim 19, wherein said detectable signal has an emission wavelength of about 450 nm.

22. The kit of claim 15, wherein said galactocerebrosidase activity is directly proportional to the production of a detectable signal.

23. The kit of claim 15, wherein said anti-catalyst is selected from the group consisting of taurocholic acid, glycine, sodium dodecylsulphate and combinations thereof.

24. The kit of claim 15, wherein said kit further comprises a buffer.

25. The kit of claim 24, wherein said buffer is selected from the group consisting of citric acid, sodium phosphate and combinations thereof.

26. The kit of claim 15, wherein said kit further comprises one ore more acids.

27. The kit of claim 26, wherein said acids are selected from the group consisting of oleic acid, taurocholic acid and combinations thereof
